(19) United States
(12) Patent Application Publication
(10) Pub. No.: US 2023/0082935 A1
(43) Pub. Date: Mar. 16, 2023

(54) IN OVO VACCINES IN COMBINATION WITH PROBIOTICS

(71) Applicant: Nutritional Health Institute Laboratories, LLC, Tallahassee, FL (US)

(72) Inventor: Konky Sotomayor, Palm Harbor, FL (US)

(73) Assignee: Nutritional Health Institute Laboratories, LLC, Tallahassee, FL (US)

(21) Appl. No.: 17/946,445

(22) Filed: Sep. 16, 2022

Related U.S. Application Data
(60) Provisional application No. 63/244,970, filed on Sep. 16, 2021.

(51) Int. Cl. A61K 39/112; A23K 10/18

(52) U.S. Cl. CPC A61K 39/0275; A23K 10/18; A61K 2039/54

(57) ABSTRACT

This invention is directed to a product for use in avian subjects comprising a combination of in ovo vaccine and probiotic, and related methods.

### CROSS-REFERENCE TO RELATED APPLICATIONS

This application claims the benefit of U.S. Provisional Patent Application No. 63/244,970, filed with the U.S. Patent and Trademark Office on Sep. 16, 2021. Said application is incorporated by reference herein in its entirety.

### FIELD OF INVENTION

This invention relates to a product for use in avian subjects comprising a combination of an in ovo vaccine and a probiotic, and related methods.

### BACKGROUND

The presence of Salmonella in commercial meat and food products is a major public health concern. Consumption of poultry products contaminated with Salmonella bacteria, such as Salmonella enteritidis or Salmonella typhimurium, is a significant source of gastrointestinal infections in humans. Such infections may lead to serious illness or, in severe cases, death. The spread of antibiotic-resistant strains in domestic flocks also gives reason for concern. Further, Salmonella infections in poultry such as chickens, turkeys, and ducks raise concerns for poultry producers due to increasing rates of morbidity and mortality as well as losses attributable to culling and/or rejection of infected birds.

Salmonella infections may be spread via intraspecies or horizontal transmission, i.e., from animal to animal, and/or via interspecies or vertical transmission, i.e., from animal to humans. Horizontal transmission of Salmonella bacteria is typically via exposure to environmental factors such as, for example, contaminated feces, bedding, nesting materials and/or other fomites. In contrast, vertical transmission of Salmonella bacteria is typically via oral exposure to the bacteria such as by handling contaminated raw meats. Vertical transmission may also occur via shell contamination and/or internal transovarian contamination of the yolk of eggs produced by infected birds.

The basis for good control of Salmonella infections in farm environments, in particular, in poultry farms, is good farming and hygiene practices. Such practices include, for example, managing and preventing contamination of feeds, monitoring of animal health, cleaning and disinfection of coops and pens, and control of pest species such as, for examples, rodents. Testing and removal of infected or pathogen-positive animals from production and/or contact with uninfected animals are also vital to controlling horizontal and/or vertical transmission of such infections.

Poultry infected with Salmonella bacteria generally develop a strong immune response to the pathogen which is typically manifested by progressive reduction in excretion of the organism and reduced disease and excretion upon subsequent challenge. Accordingly, there is a need for an effective means for inducing an immune response to Salmonella bacteria in poultry which results in reduced disease and excretion or shedding of the bacteria while reducing productivity losses attributable to culling and/or rejection of infected birds.

It is generally believed that vaccines are primarily used for public health reasons and that vaccination has limited effect on improving animal health and welfare. There is a need for an antigen composition or vaccine effective to result in improved avian health and welfare such as may be manifested by increased weight gain and reduced mortality.

Some antigens may interfere with efficacy of other vaccines or medications administered simultaneously with and/or subsequent to vaccination. Additionally or alternatively, particular antigens may interfere with or affect the accuracy of traditional test or screening tools used to detect active or prior infection. There is a demand for a Salmonella antigen which may be administered to domestic poultry and fowl without reducing the effectiveness of other vaccines such as, for example, Marek's disease vaccines. U.S. Pat. No. 7,935,355 describes vaccine antigen compositions that when inoculated in ovo in avian species induce an immune response and/or provide enhanced immunity to a pathogen such as Salmonella spp., Escherichia coli, Clostridium perfringens, or a combination thereof.

Human illness from the consumption of undercooked poultry meat may also arise from Campylobacter spp. bacteria infection in the meat, in particular C. jejuni and for instance C. coli. While Campylobacter colonize the gastrointestinal tract (e.g. intestinal mucosa) of most warm-blooded animals, the avian gastrointestinal tract, internal body temperature of 41° C., and microaerophilic environment provide optimal conditions for Campylobacter colonization. Campylobacter colonization is typically in the ceca and small intestine of chickens, however, it may become invasive, appearing in the liver, spleen, deep muscle, blood, and so forth. (Deng et al., “Current Perspectives and Potential of Probiotics to Limit Foodborne Campylobacter in Poultry” Frontiers in Microbiology (2020) doi: 10.3389/fmicb.2020.583429).

Probiotics are or include live bacteria, fungi, or yeast that supplement gastrointestinal flora and help maintain healthy digestive and immune systems, for the purposes of the present invention in avian species (including an individual avian subject), and may promote the birds' overall health and growth. Some sources suggest that probiotics may be included in poultry diets as an alternative to antibiotics. The benefits of probiotics may be outweighed or negatively impacted by poor intestinal health of the birds and by incubation conditions, feedstuff, water quality, and the bacterial strains used. (Jha et al., “Probiotics (Direct-Fed Microbials) in Poultry Nutrition and Their Effects on Nutrient Utilization, Growth and Laying Performance, and Gut Health: A Systematic Review” Animals 10:1863 (2020)). Dietary Bacillus licheniformis supplementation may enhance growth and antioxidant status and provide other benefits in Clostridium perfringens-induced necrotic enteritis in broiler chickens. (Zhou et al., “Effects of Bacillus licheniformis on the growth performance and expression of lipid metabolism-related genes in broiler chickens challenged with Clostridium perfringens-induced necrotic enteritis” Lipids in Health and Disease 15:48 (2016)). Also, B. licheniformis and Bacillus subtilis supplementation has been reported to decrease Salmonella loads in the ceca of the chicken gastrointestinal tract, potentially decreasing broiler carcass contamination with Salmonella. (Shanmugasundaram et al., J. Appl. Poult. Res. 29:808-816 (2020)). Supplementing feed with probiotics is not understood well enough for it to be used as a routine measure to limit Campylobacter colonization in chickens or other avian species. (Deng et al. (2020)).

It is desirable to minimize infection in avian species such as domestic chickens in order to minimize infection in humans. Also, reducing the use of antibiotics and improving the health of avian species is desirable.

### SUMMARY OF THE INVENTION

The present invention is directed to a combination comprising an in ovo vaccine and a probiotic composition. In an embodiment, the probiotic is in oral form. In an embodiment, the present invention comprises a kit comprising an in ovo vaccine and a probiotic composition. In an embodiment, the kit further comprises instructions. In an embodiment, a combination and/or kit of this invention and/or method thereof is for reducing or preventing infection in an avian subject or subjects and/or reducing or preventing transmission of infection to and/or from an avian. In an embodiment, the combination and/or kit promotes the health and/or growth of an avian subject or subjects, including for instance improving gut health, increasing body weight, and/or improving dietary performance.

The present invention is also a method of treating or preventing infection in an avian subject comprising the steps of providing an in ovo vaccine before hatching and administering the vaccine to the egg encasing the subject, and then, after hatching the subject from the egg, administering a probiotic composition to the subject. In an embodiment, a method of this invention promotes the health and/or growth of an avian, including for instance improving gut health, increasing body weight, and/or improving dietary performance. In addition, the present invention may be used alone or with a Marek's Disease vaccine, for instance to reduce shedding of E. coli or Salmonella bacteria.

### BRIEF DESCRIPTION OF THE DRAWINGS

FIG. 1 is a graph showing body weight gain in chickens at 7 days, 14 days, 21 days, 28 days, 35 days, and 42 days of age (for each set of 5 bars, left to right: Control group, C. jejuni infection group, C. jejuni infection+10 mg B. subtilis-B. licheniformis group, C. jejuni infection+50 mg B. subtilis-B. licheniformis group, C. jejuni infection+100 mg B. subtilis-B. licheniformis group).

FIG. 2 is a graph showing cumulative feed consumption in chickens at 7 days, 14 days, 21 days, 28 days, 35 days, and 42 days of age (for each set of 5 bars, left to right: Control group, C. jejuni infection group, C. jejuni infection+10 mg B. subtilis-B. licheniformis group, C. jejuni infection+50 mg B. subtilis-B. licheniformis group, C. jejuni infection+100 mg B. subtilis-B. licheniformis group).

FIG. 3 is a graph showing feed efficiency in chickens at 7 days, 14 days, 21 days, 28 days, 35 days, and 42 days of age (for each set of 5 bars, left to right: Control group, C. jejuni infection group, C. jejuni infection+10 mg B. subtilis-B. licheniformis group, C. jejuni infection+50 mg B. subtilis-B. licheniformis group, C. jejuni infection+100 mg B. subtilis-B. licheniformis group).

FIG. 4 is a graph showing cecal C. jejuni load in chickens at 14 days, 21 days, 28 days, 35 days, and 42 days of age (for each set of 5 bars, left to right: Control group, C. jejuni infection group, C. jejuni infection+10 mg B. subtilis-B. licheniformis group, C. jejuni infection+50 mg B. subtilis-B. licheniformis group, C. jejuni infection+100 mg B. subtilis-B. licheniformis group).

FIG. 5 is a graph showing villi height in chickens at 14 days, 21 days, 28 days, 35 days, and 42 days of age (for each set of 5 bars, left to right: Control group, C. jejuni infection group, C. jejuni infection+10 mg B. subtilis-B. licheniformis group, C. jejuni infection+50 mg B. subtilis-B. licheniformis group, C. jejuni infection+100 mg B. subtilis-B. licheniformis group).

FIG. 6 is a graph showing crypt depth in chickens at 14 days, 21 days, 28 days, 35 days, and 42 days of age (for each set of 5 bars, left to right: Control group, C. jejuni infection group, C. jejuni infection+10 mg B. subtilis-B. licheniformis group, C. jejuni infection+50 mg B. subtilis-B. licheniformis group, C. jejuni infection+100 mg B. subtilis-B. licheniformis group).

FIG. 7 is a graph showing villi height:crypt depth ratio (VH:CD ratio) in chickens at 14 days, 21 days, 28 days, 35 days, and 42 days of age (for each set of 5 bars, left to right: Control group, C. jejuni infection group, C. jejuni infection+10 mg B. subtilis-B. licheniformis group, C. jejuni infection+50 mg B. subtilis-B. licheniformis group, C. jejuni infection+100 mg B. subtilis-B. licheniformis group).

FIG. 8 is a graph showing bile anti-C. jejuni IgA in chickens at 21 days, 28 days, 35 days, and 42 days of age (for each set of 5 bars, left to right: Control group, C. jejuni infection group, C. jejuni infection+10 mg B. subtilis-B. licheniformis group, C. jejuni infection+50 mg B. subtilis-B. licheniformis group, C. jejuni infection+100 mg B. subtilis-B. licheniformis group).

FIG. 9 is a graph showing serum anti-C. jejuni IgG in chickens at 21 days, 28 days, 35 days, and 42 days of age (for each set of 5 bars, left to right: Control group, C. jejuni infection group, C. jejuni infection+10 mg B. subtilis-B. licheniformis group, C. jejuni infection+50 mg B. subtilis-B. licheniformis group, C. jejuni infection+100 mg B. subtilis-B. licheniformis group).

### DETAILED DESCRIPTION OF THE INVENTION

The present invention combines an in ovo vaccine and a probiotic composition, and is for administration in an avian species (including an avian subject and/or one or more such as a group of avian subjects, such as a flock). In an embodiment, this combination treats and/or prevents infection such as by Salmonella spp. and optionally Campylobacter spp. and/or Clostridium spp. or other pathogenic bacteria (and/or other microorganisms) in an avian subject. For instance, the combination of in ovo vaccine and probiotic composition may decrease the amount of e.g. pathogenic or otherwise undesirable bacteria and/or other microorganism(s) in the subject, decrease bacterial and/or other microorganism proliferation, decrease symptoms of infection, and/or protect the avian subject from Salmonella infection as well as infection from other pathogens such as infection from Campylobacter bacteria (e.g. as campylobacteriosis) and Clostridium bacteria (e.g. as necrotic enteritis).

In an embodiment, this combination promotes health and/or growth of the subject, for instance by improving one or more parameters of good health including good gut health, increasing body weight of the subject, and improving dietary performance and so extending profitable food conversion of the subject. In an embodiment, this combination avoids or reduces the need for antibiotic administration to the avian animal. In an embodiment, the combination of in ovo vaccine and probiotic composition prevents or reduces infection from or of other animals. In an embodiment, the combination of vaccine and probiotic according to this invention is a synergistic combination providing one or more synergistic effects.

The present invention is also a method of treating or preventing infection in an avian subject comprising the steps of providing an in ovo vaccine before hatching and administering the vaccine to the egg encasing the subject, and then, after hatching the subject from the egg, administering a probiotic composition to the subject. In an embodiment, the present invention comprises a method of “boosting” (improving) immune system response of an avian subject with the combination of an in ovo vaccine and probiotic. In an embodiment, said improvement or “boost” is synergistic, improving the immune system response of the subject (and/or a group of subjects) in a statistically significant manner, for instance over that of the in ovo vaccine alone and/or probiotic alone (and placebo alone). In an embodiment, reference to a synergistic method of the present invention may be to synergy in an individual subject and/or in a group of 2 or more subjects, including for instance a flock or other grouping of birds; in an embodiment, with synergy supported by statistically significant differences from vaccine and/or probiotic alone.

The below definitions and discussion are intended to guide understanding but are not intended to be limiting with regard to other disclosures in this application. References to percentage (%) in compositions of the present invention refers to the % by weight of a given component to the total weight of the composition being discussed, also signified by “w/w”, unless stated otherwise. References to “comprising” may also construed as supporting narrower embodiments of the present invention, for instance within the scope of transitional phrases such as “consisting essentially of” or “consisting of”, or their like.

In the present invention, a “product” refers to a combination of an in ovo vaccine and a probiotic of this invention, and in an embodiment, may be referred to as an “immunity product”. In an embodiment, said product and combination according to this invention is synergistic, providing “boosted” (improved) immune response in one or more avian subjects over the vaccine alone or the probiotic alone. An improved immune response may be identified by decreased fecal bacterial count (e.g. Salmonella, E. coli), decreased lesions/lesion formation, improved immune system response such as increased or otherwise improved antibody production, and/or other measurable or apparent improvements. In an embodiment, a product of this invention may be an in ovo vaccine as described throughout this application, or a probiotic composition.

In the present invention, a “vaccine” comprises, consists essentially of, or consists of an antigen composition that may be administered in ovo. In an embodiment, the vaccine is for stimulating an immune response in an avian species (e.g. an avian subject and/or a more than one avian subjects, such as a flock) to at least one pathogenic organism (e.g. an intestinal pathogen) such as Salmonella species, such as for instance S. enterica (e.g. S. typhimurium, S. agona, S. Kentucky, and/or S. enteritidis); and/or Campylobacter species, such as for instance C. jejuni. In an embodiment, said stimulating immune response provides protection to the subject and/or group/flock of subjects from infection by the organism; in an embodiment, said protection extends to infection by other organisms as well. In an embodiment, a vaccine of this invention is an in ovo vaccine, to be administered in ovo. In an embodiment, a vaccine against Salmonella infection of this invention comprises E. coli, S. enteritidis, S. agona, S. Kentucky, S. typhimurium, Pseudomonas aeuroginosa, and Aerobacter aerogenes, for instance as described in Examples A and B.

In an embodiment, an antigen composition is a Salmonella (e.g. multivalent) antigen composition comprising (or consisting essentially of or consisting of) at least one Salmonella enterica subspecies. In an embodiment, said at least one S. enterica subspecies is in O-serogroup B, at least one Salmonella enterica subspecies is in O-serogroup C₃, and at least one Salmonella enterica subspecies is in O-serogroup D. In an embodiment, the composition induces an immune response in an inoculated avian species (including for instance an individual subject or more than one subject, such as a flock) to at least one intestinal pathogenic organism. The Salmonella enterica subspecies may comprise naturally-occurring wild strains from O-serogroups B, C₃, and/or D. In an embodiment, a vaccine of this invention may comprise about 40-50%, such as 46%, Salmonella enterica subspecies in O-serogroup B, about 25-35%, such as 31%, Salmonella enterica subspecies in O-serogroup D, and about 20-23%, such as about 23%, Salmonella enterica subspecies in O-serogroup C₃. In an embodiment, Salmonella enterica subspecies e.g. in O-serogroup B may comprise Salmonella typhimurium, Salmonella agona and/or combinations thereof. In an embodiment, the Salmonella enterica subspecies in O-serogroup B may comprise ATCC strain 14028 of Salmonella typhimurium. Salmonella enterica subspecies in O-serogroup C₃ may comprise Salmonella Kentucky. Salmonella enterica subspecies in O-serogroup D may comprise Salmonella enteritidis such as, for example, ATCC Strain 13076 of Salmonella enteritidis. In an embodiment, a vaccine according to this invention may further comprise other components such as preservatives (including e.g. antibiotics and/or antifungal agents), antibiotics (e.g. gentamycin), antifungal agents (e.g. nystatin), water, buffer, culture medium, physiologically acceptable carriers, physiologically acceptable diluents, stabilizers such as SPGA, carbohydrates (e.g. sorbitol, mannitol, starch, sucrose, dextran, glutamate, glucose), proteins (e.g. dried milk serum, albumin, casein), alcohols, polyols, and other substances as needed, preferred, or desired.

A “bacterin” or “bacterin vaccine” according to this invention refers to a vaccine composition generally comprised of dead or inactivated bacteria species. Inactivation may be achieved for instance by heat, chemical agents such as methanol, acetone, ethanol, acetic acid, formalin, carbon dioxide, or others; irradiation such as UV light; alkaline or acidic environment for instance pH≥10, 11, or 12, or pH≤3 or 4; sonication; and other techniquies for instance as known in the art. In an embodiment, a vaccine of the present invention is a vaccine such as a bacterin vaccine comprising about 40-50%, e.g. 46%, Salmonella enterica subspecies in O-serogroup B, about 25-35%, e.g. 31%, Salmonella enterica subspecies in O-serogroup D, and about 20-30%, e.g. 23%, Salmonella enterica subspecies in O-serogroup C₃. Subspecies in O-serogroup B may comprise Salmonella typhimurium, Salmonella agona and/or combinations thereof. Subspecies in O-serogroup C₃ may comprise Salmonella Kentucky. Subspecies in O-serogroup D may comprise Salmonella enteritidis such as, for example, ATCC strain 13076. In an embodiment, a bacterin vaccine of this invention comprises or consists of ATCC strain 13076 of Salmonella enteritidis, ATCC strain 14028 of Salmonella typhimurium, Salmonella agona, and Salmonella Kentucky.

In an embodiment, heat is used to prepare a bacterin vaccine of this invention such as an inactivated vaccine. In an embodiment, a vaccine of this invention uses temperatures effective to inactivate antigenic components of the vaccine such as Salmonella and other bacteria described herein. In an embodiment, heat inactivation is applied to each bacterial strain prior to mixing the strains together in the vaccine. In an embodiment, heat inactivation is applied to the vaccine after the strains are mixed together. In an embodiment, heat inactivation is before, during, and/or after preparation of a vaccine of this invention. In an embodiment, heat is applied at a temperature of 65° C. or more, 70° C. or more, 75° C. or more, 80° C. or more, 85° C. or more, 90° C. or more, 95° C. or more, 100° C. or more, 105° C. or more, 110° C. or more, 115° C. or more, 120° C. or more, 125° C. or more, 130° C. or more, including for instance heat in the range of 68-125° C. In an embodiment, heat is applied at about 115-125° C., or 120-125° C., for instance 121° C., for 1, 2, 3, 4, or 5 cycles of 5, 10, 15, 20, 25, 30, 35, 40, 45, 50, 55, or 60 minutes, including for instance two 25-45 minute cycles or one 25-45 minute cycle (for instance 35 minute cycle(s)) when preparing a vaccine of this invention to inactivate the bacteria therein. In an embodiment, heat is applied for one cycle and applied in a second or subsequent cycles if viable bacteria (for instance 1%, 0.1%, 0.01%, etc., or the like, for instance per governmental or preferred regulatory standards) remain. In an embodiment, 90% or more bacteria are inactivated in a vaccine of this invention; in an embodiment; 92.5% or more; in an embodiment, 95% or more; in an embodiment, 97.5% or more; in an embodiment, 99% or more; in an embodiment 99.9% or more, up to 100%, of bacteria in a vaccine of this invention are inactivated.

In an embodiment, an in ovo vaccine of this invention reduces a concentration of at least one pathogenic organism in a gastrointestinal tract of an inoculated avian species (such as an avian subject or more than one subject including for instance a flock). In an embodiment, a method of the present invention boosts the immune system of an avian subject and/or group of subjects, and comprises the steps of administering an in ovo vaccine and administering a probiotic. In an embodiment, an in ovo vaccine of this invention comprises a vaccine such as described throughout this invention (for instance as in Table 5), for instance a bacterin vaccine, and also comprises a Marek's disease vaccine. In an embodiment, a vaccine such as described throughout this invention (for instance as in Table 5) is administered separately from a Marek's disease vaccine. In an embodiment, the Salmonella (e.g. multivalent) antigen composition may comprise Salmonella enterica subspecies from O-serogroups B, C₃, and D which in combination stimulate an immune response in an inoculated avian species (such as an individual subject or more than one individual including a flock) to at least one intestinal pathogenic organism such as, for example, Clostridium perfringens, Salmonella species and/or Escherichia coli.

In an embodiment, a Salmonella (e.g. multivalent) antigen composition of this invention is administered alone and/or in combination with one or more other bacterial strains, such as, for example, E. coli, Pseudomonas aeruginosa, and/or Aerobacter aerogenes, and/or other poultry vaccine compositions such as, for example, a Marek's disease vaccine. Further, the Salmonella (e.g. multivalent) antigen composition may be provided and/or utilized as live or bacterin vaccine alone or in combination with other bacterial strains and/or poultry vaccine compositions.

In an embodiment, the Salmonella (e.g. multivalent) antigen composition of this invention may be administered as an in ovo antigen vaccine. For example, the Salmonella (e.g. multivalent) antigen composition, such as in the form of a bacterin vaccine, may be utilized in a combination and/or method of this invention, for instance for reducing the transmission of pathogenic gastrointestinal organisms that comprises inoculating an avian species (e.g. individual subject or more than one such as a group/flock) in ovo for instance at about 18 days embryonic age with the (e.g. bacterin) vaccine and then administering probiotics to the subject(s).

In an embodiment, an antigen composition of this invention may be included in an in ovo (e.g. bacterin) vaccine comprising: an (e.g. bacterin) vaccine comprising ATCC strain 13076 of Salmonella enteritidis, ATCC 14028 of Salmonella typhimurium, Salmonella agona, and Salmonella Kentucky; and a Marek's disease vaccine such as, for example, an HVT vaccine, a SB-1 vaccine and/or a combination thereof. The in ovo bacterin vaccine reduces a concentration of at least one pathogenic organism in a gastrointestinal tract of an inoculated avian subject. Such in ovo bacterin vaccine may be administered, in an embodiment, to the avian subject at for instance about 18 day's embryonic age.

In accordance with another embodiment, the Salmonella (e.g. multivalent) antigen composition forms an integral part of an (e.g. multivalent) antigen composition that comprises other and/or additional bacterial strains and/or poultry vaccine compositions. For example, the (e.g. multivalent) antigen composition may include seven (e.g.) field strains of bacteria such as E. coli, Pseudomonas aeruginosa, Aerobacter aerogenes, Salmonella enteritidis, Salmonella typhimurium, Salmonella agona, and Salmonella Kentucky. In an embodiment, only Salmonella bacteria such as Salmonella enteritidis, typhimurium, agona, and Kentucky, are included in a vaccine of this invention. In an embodiment, the in ovo vaccine of the present invention comprises Salmonella enterica subspecies enterica serovar Enteritidis (Salmonella enteritidis), Salmonella enterica subsp. Enterica serovar Typhimurium (Salmonella typhimurium), Salmonella enterica subsp. Enterica serovar Kentucky (Salmonella Kentucky), Salmonella enterica subsp. Enterica subsp. Enterica serovar Agona (Salmonella agona), Pseudomonas Aeruginosa, Aerobacter Aerogenes AHP462/PTA-11661, and Eschericia coli isolate #7; in an embodiment, said vaccine includes components as described in Table 5. In an embodiment, the (e.g. multivalent) antigen or antigen composition stimulates an immune response to an intestinal pathogenic organism selected from Clostridium perfringens, Salmonella spp., E. coli or a combination thereof. Such immune response may be manifested as a reduction in fecal bacterial counts for a particular pathogen such as, for example, reduction in Salmonella spp. fecal bacteria counts and/or E. coli fecal bacterial counts. Such immune response may additionally or alternatively be manifested as a reduction in lesion formation upon exposure to Clostridium perfringens.

In an embodiment, various strains of E. coli bacteria may be included in the antigen composition. Such strains of E. coli bacteria may be selected from e.g. ATCC strain 25922, a University of Delaware field isolate, one or more Delmarva field isolates, or a combination thereof. In accordance with one embodiment, the antigen composition comprises seven field strains of E. coli bacteria including ATCC strain 25922, a University of Delaware field isolate, and five Delmarva field isolates.

In an embodiment, the antigen composition comprises about 5-70%, for instance 5-10% or 65-70%, for instance 6% or 67%, of E. coli bacteria including for instance one to seven strains of E. coli bacteria. In an embodiment, each strain of E. coli bacteria is present in an approximately equal amount.

In an embodiment, various strains of Pseudomonas aeruginosa are suitable for use in the antigen composition of a vaccine of the invention. In accordance with one embodiment, the antigen composition may include ATCC strain 27653 of Pseudomonas aeruginosa. In an embodiment, the antigen composition may include about 1-40%, for instance about 10-30%, or for instance about 10%, 20%, 25%, 26%, or other amount of Pseudomonas aeruginosa.

In an embodiment, the antigen composition further comprises Aerobacter aerogenes such as in an amount of about 1-30%, for instance about 5-20%, or for instance about 8%, 9%, 10%, 11%, 12%, or other amount. In accordance with certain aspects of the invention, the antigen composition is or should be free or devoid of Enterobacter aerogenes and/or Klebsiella pneumoniae, e.g. free of detectable amounts of such; 0%.

In an embodiment, the antigen composition comprises at least four strains of Salmonella species. In an embodiment, the antigen composition comprises Salmonella enteritidis, Salmonella typhimurium, Salmonella agona, and Salmonella Kentucky. In an embodiment, the antigen composition includes ATCC strain 13076 of Salmonella enteritidis and/or ATCC strain 14028 of Salmonella typhimurium. In an embodiment, the antigen composition comprises about 1-25%, for instance about 2-12%, such as about 2%, 3%, 4%, 5%, 9%, et al., of Salmonella enteritidis; about 1-25%, for instance about 2-12%, such as 2%, 3%, 4%, 5%, 9%, 24%, et al. of Salmonella typhimurium, about 1-25%, for instance about 2-12%, such as about 2%, 3%, 4%, 5%, 9%, et al. of Salmonella agona, and about 1-25%, for instance about 2-12%, such as about 2%, 3%, 4%, 5%, 9%, 15%, et al. of Salmonella Kentucky. In an embodiment, the antigen composition includes about 4% S. enteritidis, about 3% S. typhimurium, about 3% S. agona, and about 3% S. Kentucky. In an embodiment, the antigen composition is as described in Tables described in the Examples.

In an embodiment, the antigen composition and in ovo vaccine dose comprises about 4-100% Salmonella spp., including for instance about 40-80% of total CFU/dose (activated or bacterin/inactivated), for instance about 50-70%, about 55-60%, about 56%, 57% (as shown in Table 5), or 58%. In an embodiment, other bacteria such as E. coli, A. aerogenes, and/or P. aeruginosa comprise the remainder of bacteria in an antigen composition of this invention (100%-Salmonella content), including for instance about 30-50%, such as 43% as shown in Table 5 (6% E. coli, 11% A. aerogenes, 26% P. aeruginosa). In an embodiment, a vaccine dose of according to this invention is a range between the amounts provided in Table 1 (far right columns) for lower dosage and higher dosage preparations. In an embodiment, a final vaccine dose of the present invention (e.g. a bacterin vaccine) includes about 10⁸-10¹¹ CFU Salmonella Kentucky, about 10⁸-10¹¹ CFU Salmonella typhimurium, about 10⁸- 10¹¹ CFU Salmonella agona, and about 10⁸-10¹¹ CFU Salmonella enteritidis. In an embodiment, said final vaccine dose further comprises about 10⁸-10¹¹ CFU A. aerogenes, about 10⁸-10¹¹ CFU P. aeruginosa, and/or about 10⁸ to about 10¹³ E. coli. In an embodiment, a “high dose” of one or more vaccine bacterial components includes the amounts as marked throughout the application for instance in Table 1 and for instance as described immediately above, in the upper half of the above-noted ranges. In an embodiment, a “low dose” of one or more vaccine bacterial components includes the amounts as marked throughout the application for instance in Table 1 and for instance as described immediately above, in the lower half of the above-noted ranges. In an embodiment, a stock preparation according to the present invention may comprise one or more of: about 10⁸-10¹⁴ CFU Salmonella Kentucky, about 10⁸-10¹⁴ CFU Salmonella typhimurium, about 10⁸-10¹⁴ CFU Salmonella agona, and/or about 10⁸-10¹⁴ CFU Salmonella enteritidis; in an embodiment, the above ranges may be applied to any Salmonella enteritis or to Salmonella spp. In an embodiment, said final vaccine dose further comprises about 10⁸-10¹⁴ CFU A. aerogenes, about 10⁸- 10¹⁴ CFU P. aeruginosa, and/or about 10⁸ to about 10¹⁴ E. coli.; including for instance the stock preparation(s) described in Table 1. The above ranges and definitions are not intended as limiting; amounts may vary.

In an embodiment, the (e.g. multivalent) antigen or antigen composition may be utilized as or in an in ovo vaccine for inoculating an avian subject or subjects, including for instance a flock. For example, about 0.005 ml to about 0.05 ml of the (e.g. multivalent) antigen or antigen composition may be used to inoculate an embryonated egg. In an embodiment, the (e.g. multivalent) antigen or antigen composition may be given in a dose of about 0.0063 ml to about 0.0375 ml per embryonated egg. Other amounts may be provided depending on the avian species and other relevant variables.

In an embodiment, the (e.g. multivalent) antigen or antigen composition is suitable for use alone as a bacterin vaccine or in combination with one or more other vaccine preparations. For example, the antigen composition may be administered sequentially with or simultaneously with another vaccine preparation such as, for example, a Marek's Disease vaccine.

In accordance with one embodiment, the antigen composition may be mixed or combined with a Marek's Disease vaccine. Such combined or mixed vaccine comprises a bacterin vaccine including seven strains of E. coli, Pseudomonas aeruginosa, Aerobacter aerogenes, Salmonella enteritidis, Salmonella typhimurium, Salmonella agona, and Salmonella Kentucky and a Marek's Disease vaccine. The Marek's Disease vaccine may include an HVT vaccine, a SB-1 vaccine, or a bivalent vaccine including a mixture or combination of HVT and SB-1 strains. In an embodiment, the bacterin and Marek's Disease vaccine may be combined in any suitable ratio. For example, the combined vaccine may have a bacterin vaccine to Marek's Disease vaccine ratios in the range of about 1:15 to 15:1, including for instance 1:10 to 10:1, 1:5 to 5:1, 1:3 to 3:1, 1:2 to 2:1, or 1:1. In an embodiment, the bacterin vaccine and the Marek's Disease vaccine may be combined in a 1:1 ratio.

The combined bacterin-Marek's Disease vaccine reduces a concentration of at least one pathogenic organism in a gastrointestinal tract of an inoculated avian subject. Such pathogenic organism may include E. coli, Salmonella spp. or a combination thereof.

In an embodiment, the combined bacterin-Marek's Disease vaccine may be an in ovo vaccine suitable for inoculating an avian subject. For example, the combined bacterin-Marek's Disease vaccine may be administered in ovo in a dose of about 0.005 ml to about 0.1 ml combined vaccine per embryonated egg. In an embodiment, a dose of the combined bacterin-Marek's Disease vaccine may include about 0.0063 ml to about 0.0375 ml bacterin vaccine.

In addition to the above, in an embodiment, an in ovo vaccine of the present invention is as described in U.S. Pat. No. 7,935,355. U.S. Pat. No. 7,935,355 is incorporated by reference herein to the extent permitted by law for the purpose of describing antigen compositions and in ovo vaccines of this invention, including for instance the Examples of the '355 patent.

In the present invention, a “probiotic composition” refers to a composition (such as a symbiotic composition) comprising live bacteria, fungi, or yeast that help maintain healthy digestive and immune systems in avian species, including an avian subject and/or a group of avian subjects, such as a flock. In an embodiment, a probiotic composition supplements gastrointestinal flora in an avian subject and may promote the subject's overall health and growth. In an embodiment, a probiotic composition of this invention is to boost and/or enhance protection provided by the vaccine; and vice versa; for instance synergistically. In an embodiment, a probiotic of this invention comprises 25-30 billion CFU/ml or more of all or some of the following bacteria: Bacillus amyloliquefaciens, B. licheniformis, B. subtilis, B. pumulis, Lactobacillus reuteri, L. acidophilus, L. rhamnosus, Saccharomyces cerevisiae. The consortia of bacteria may change. In an embodiment, a probiotic of this invention administers 5-15 million, for instance 7-12 million, for instance 9-11 million, for instance, 10 million total CFU (e.g. activated or inactivated, bacterin) per dose per chicken. In an embodiment, a probiotic composition of this invention comprises Bacillus subtilis, for stock or administration, for instance in an amount of about 10⁶-10¹⁰, or for instance about 10⁶-10¹² CFU, or any amount therebetween. In an embodiment, a probiotic composition of this invention comprises Bacillus licheniformis, for stock or administration, for instance in an amount of about 10⁶-10¹⁰, or for instance about 10⁶-10¹² CFU, or any amount therebetween. In an embodiment, a probiotic composition of this invention comprises C. jejuni, for stock or administration, for instance in an amount of about 10⁶-10¹⁰, or for instance about 10⁶-10¹² CFU, or any amount therebetween. In an embodiment, a probiotic composition of this invention comprises Bacillus amyloliquefaciens, B. licheniformis, B. subtilis, B. pumulis, Lactobacillus reuteri, L. acidophilus, L. rhamnosus, and/or Saccharomyces cerevisiae, and/or other probiotic microbial (e.g. bacteria, yeast) component according to the present invention, for stock or administration, for instance in an amount of about 10⁶-10¹⁰, or for instance about 10⁶-10¹² CFU, or any amount therebetween. In an embodiment, a probiotic composition is as described in Example B, C, or D of this application. In an embodiment, a probiotic composition of this invention comprises an effective amount of at least one of Bacillus amyloliquefaciens, Bacillus licheniformis, Bacillus subtilis, Bifidobacterium brevis, Bifidobacterium bifidum, Bifidobacterium longum, Lactobacillus reuteri, Lactobacillus acidophilus, Lactobacillus rhamnosus, Saccharomyces boulardii, and/or Streptococcus spp., for instance in an amount of 10⁶-10¹⁰ CFU, or for instance about 10⁶-10¹² CFU, or any amount therebetween. In an embodiment, a probiotic composition of this invention is administered orally to an avian subject. In an embodiment, a probiotic composition of this invention is a dietary supplement, for instance in solid form such as an oral feed such as a solid dry formula, or in liquid form such as a water-soluble probiotic to be used by dissolving the probiotic in drinking water. In an embodiment, a probiotic of this invention includes additional substances, which may be formulated with the probiotic or administered with the probiotic, said additional substances including nutrients to support beneficial microbe growth and production of fermentation metabolites. In an embodiment, said probiotic and/or nutrients are added at a rate and/or amount of about 0.5-2%, for instance 1%, of the avian subject drinking water or dry feed throughout chicken growth to sustain the population of beneficial microbes in the gut. In an embodiment, a “probiotic” of this invention may further comprise additional ingredients as desired, including for instance additional nutrients to support beneficial microbe growth and production of fermentation metabolites.

A “subject” according to this invention is avian (a bird), including for instance a wild bird, poultry, fowl such as domestic fowl, chicken such as a broiler or roaster chicken, layers, breeders, hens, roosters, duck, turkey, goose, guinea fowl, squab, game hen, pheasant, quail, ostrich, grouse, domesticated or wild birds, swans, pigeons, doves, game birds. The present invention may apply to an individual subject or to one or more subjects, including a group of 2-10, 10-20, 20-30, 30-50, 50-100, 100-200, 200-500, 500-1000, 1000-10,000, or more (including any number within or defined by these ranges). Such a group may be a flock. A group or “flock” may be of the same avian species or subspecies or other categorization, or may include e.g. more than one avian species or subspecies. The term “flock” is not intended as limiting to a given avian species or subspecies or the like; “flock” is intended to include any avian (bird) species for the purposes of this invention. Reference to “a subject” according to this invention may refer to 1 subject and/or to at least 1 subject (i.e. 2 or more subjects), unless expressly indicated otherwise.

In the present invention, “administering”, “administration”, and the like refer to providing a combination of in ovo vaccine and probiotic of the present invention to a subject (one or more than one), each in an effective amount so that the vaccine and probiotic (or components thereof) reach the subject's bloodstream (vaccine) and gastrointestinal tract (probiotic) and/or the subject's other tissues and/or the subject's cells and act thereon to prevent and/or reduce infection in the subject such as Salmonella infection and optionally Campylobacter, Clostridium, or other infection and in an embodiment promote the subject's health and/or growth. Administration may be by the subject or by another. Administration of a vaccine according to this invention is in ovo, before hatching a chick from the egg; typically, in ovo vaccination is via injection (for instance by known techniques) 17-18 days after the egg is laid (embryonic “E” stage), however, vaccination may occur prior to or after E17-18 days, even after hatching if desired. Administration of a probiotic is after hatching from the egg and is enteral, preferably oral, via the gastrointestinal tract of the chicken. In an embodiment, administration of probiotic according to this invention is daily, in an embodiment is for a period of 1-56 days, and may begin as early as the animal's first drinking or feeding upon hatching, including for instance from hatch to three days of age, or during or throughout avian growth period(s). In an embodiment, dosing of a probiotic is every 6 to 8 hours every 1, 2, 3, 4, or 5 days, for instance every 3 days, for instance on hatching day (D0) for three days or throughout a growth period, to sustain the population of beneficial microbes in the gut. In an embodiment, administration of a probiotic according to this invention is oral, for instance in the form of a dietary supplement, and/or for instance in liquid form such as in water or in solid form such as in chicken feed. In an embodiment, a probiotic of the present invention is administered in water or in solid form, but not both. Other forms may include for instance pills, tablets, time release formulations, osmotic controlled release formulations, solutions, softgels, suspensions, emulsions, syrups, elixirs, tinctures, gels, ointments, suppositories, enemas, Murphy drips, and so forth. In an embodiment, administration may also be by other physiologically acceptable routes.

In an embodiment, a method of treating or preventing Salmonella and optionally Campylobacter, Clostridium, and/or other infection in an avian subject of the present invention comprises the steps of providing an in ovo vaccine and a probiotic composition of the present invention and administering the in ovo vaccine and probiotic composition to the subject to treat Salmonella infection and optionally Campylobacter infection, Clostridium infection, and/or other infection (e.g. reduce amount of bacteria present or slow or halt Salmonella and/or Campylobacter and/or Clostridium or other infectious growth) and/or prevent Salmonella infection and optionally Campylobacter infection and/or Clostridium infection (e.g. preventing Salmonella, Campylobacter, and/or Clostridium growth in the subject or preventing Salmonella and optionally Campylobacter and/or Clostridium entry or colonization) in the subject.

A “kit” according to the present invention comprises a combination of an in ovo vaccine and a probiotic composition of the present invention; in an embodiment, a synergistic combination. In an embodiment, the vaccine and probiotic composition are included together in a single package; in an embodiment, the vaccine and probiotic composition are packaged separately. In an embodiment, instructions are included in the package comprising vaccine and probiotic composition, and/or instructions are included with the vaccine and/or the probiotic composition. Instructions may be physically printed on paper or the like or for instance associated with an internet link identified in the kit. In an embodiment, a kit of this invention may further include an assay or other test for evaluating infection of a subject according to this invention (for instance by testing fecal matter, saliva, blood, or other bodily substances of a subject); an assay or other test for measuring body weight of a subject and/or a parameter associated with health of the subject, preferably also accompanied by a predetermined standard value for an average healthy subject and instructions for determining whether the subject's health has improved compared with its own previous assessment or compared with an average healthy subject after the administration of the combination of the present invention; and/or an assay or other test to assess the level of probiotics administered according to this invention in the gastrointestinal system of the subject.

The present invention may be further understood in connection with the below Examples and with embodiments described throughout this application. The following non-limiting Examples and embodiments described below and throughout this application are provided to illustrate the invention.

### EXAMPLES

### Example A

### Formulation and Preparation of High Salmonella-MDV Combination Vaccine

The preparation of a High Dose Salmonella Vaccine of this invention is described in this Example.

A 2× High Salmonella Vaccine, 2×MDV (Marek's disease virus vaccine) vaccine are prepared, and then further prepared and packaged into the 1× High Salmonella-MDV Combination Vaccine of this invention.

### Materials

Heat-inactivated bacterial vaccine stock (Double-autoclaved), preserved in refrigerator
TSB (Tryptic Soy Broth): For growing each bacterial strain
TSA plates (Tryptic Soy Agar plates): for characterization of each bacterial strain before mixing
DPBS (Dulbecco's Phosphate Buffered Saline): for formulating the final vaccine
Gentamycin (50 mg/ml): for formulating the final vaccine
Nystatin (10,000 U/ml): for formulating the final vaccine
MDV (Marek's disease virus vaccine), preserved in liquid nitrogen
   As discussed further below, the MDV is to be prepared as 2×MDV vaccine and added 1:1 to the 2× High Salmonella Vaccine at the day of inoculation to produce a final 1× High Salmonella-MDV combination vaccine.

### Procedures

Prepare 2× High Salmonella Vaccine.
a. Briefly, each bacterial strain shown in Table 1 was grown in TSB, harvested, characterized, and tested for quality control purposes. The strain was then counted and double-autoclaved followed by mixing to prepare a 2× High Salmonella Vaccine as shown in Table 1. The 2× High Salmonella Vaccine is to be mixed 1:1 with 2×MDV vaccine at the day of inoculation to produce a final 1× Salmonella-MDV combination vaccine.
b. Mixing of the 2× High Salmonella Vaccine with the 2×MDV vaccine on the day of inoculation is dependent on the result of the sterility testing of the High Salmonella Vaccine (Salmonella vaccine must pass sterility testing requirements to be mixed).

TABLE 1
Concentration of each bacterial stock and preparation of High Dose Salmonella Vaccine

| | | | | | | | | | Final | | | |
|---|---|---|---|---|---|---|---|---|---|---|---|---|
| | | | | | | | 1X Final | | higher dosage | | | |
| | | | | | | | concentration | | concentration | | | |
| | | | Volume of | | Resulting 2X | | per ml in | | per 0.1 ml | | Example of | |
| | Stock vaccine | | stock vaccine | | concentration | | final vaccine | | dose (100-fold | | lower dosage | |
| Bacterial | concentration | | required for | | per ml (to be | | (after mixing | | increase (10²) | | concentration | |
| isolate/ | (CFU/ml or | | 2X dose prep | | diluted 1:1 | | 1:1 with 2X | | than lower | | prep. per 0.1 | |
| Preservative | as noted) | | (ml or as noted) | | with 2X MDV) | | MDV) | | dosage prep) | | ml dose | |
| S. Kentucky | 5 × 10¹² | | 6 | | 6 × 10¹¹ | | 3 × 10¹¹ | | 3 × 10¹⁰ | | 3 × 10⁸ | |
| S. typhimurium | 9 × 10¹² | | 5.3 | | 9.6 × 10¹¹ | | 4.8 × 10¹¹ | | 4.8 × 10¹⁰ | | 4.8 × 10⁸ | |
| S. agona | 3 × 10¹² | | 5.6 | | 3.4 × 10¹¹ | | 1.7 × 10¹¹ | | 1.7 × 10¹⁰ | | 1.7 × 10⁸ | |
| S. entertidis | 2.5 × 10¹² | | 7.2 | | 3.6 × 10¹¹ | | 1.8 × 10¹¹ | | 1.8 × 10¹⁰\| | | 1.8 × 10⁸ | |
| E. coli | 1.7 × 10¹⁴ | | 7 | | 2.4 × 10¹³ | | 1.2 × 10¹³ | | 1.2 × 10¹²\| | | 1.2 × 10¹⁰ | |
| A. aerogenes | 3 × 10¹² | | 7.3 | | 4.4 × 10¹¹ | | 2.2 × 10¹¹ | | 2.2 × 10¹⁰ | | 2.2 × 10⁸ | |
| P. aeruginosa | 8 × 10¹² | | 6.5 | | 1.04 × 10¹² | | 5.2 × 10¹¹ | | 5.2 × 10¹⁰\| | | 5.2 × 10⁸ | |
| Gentamycin | 50 | mg/ml | 60 | ul | 60 | ug/ml | 30 | ug/ml | 3 | ug | 3 | ug |
| | (stock) | | | | | | | | | | | |
| Nystatin | 10,000 | U/ml | 300 | ul | 60 | U/ml | 30 | U/ml | 3 | U | 3 | U |

| | (stock) | | |
|---|---|---|---|
| DPBS (dilute) | NA | 4.74 | |
| Total | NA | 50 | ml |

### Packaging

All vaccine vials to be prepared and filled in the hood and with a final volume of 10 ml/vial, sufficient to provide a 0.1 ml dose/fertile egg to 100 eggs.

Packaging begins with filling the 10 ml vials with 5 ml of 2× High Salmonella Vaccine (contains all strains as in Table 1).

3 vials prepared at the beginning, middle, and end of packaging were submitted for 14-day sterility testing.

An additional 5 ml of 2×MDV vaccine is to be added on the day of inoculation to each vaccine vial (total final volume 10 ml/vial). MDV vaccine is also to pass sterility testing before mixing with 2× High Salmonella Vaccine.

Placebo vials are to be prepared at the time of inoculation and to include DPBS in addition to Nystatin and Gentamycin at the same concentration used in the Salmonella vaccine.

### Example B

### Efficacy and Protection Against Salmonella Challenge with High Dose Salmonella-MDV Combination Vaccine (Heat Inactivated in Ovo Vaccine) and Probiotic Treatment

This Example will show the efficacy of an inactivated Salmonella vaccine against a Salmonella challenge when administered in combination with a probiotic in-water treatment, where said probiotic is to boost/enhance protection provided by the vaccine.

This Example will test the ability of an in ovo inactivated Salmonella vaccine administered with a Marek's Disease Vaccination and in conjunction with probiotics in drinking water to provide protection against a subsequent Salmonella challenge.

TABLE 2
Study Design

| | | | | | | # of Chicks | |
|---|---|---|---|---|---|---|---|
| | | Age at | Amount | | # of Eggs | Enrolled | # of Chicks |
| Group# | Treatment | Administration | or Rate | Route | Injected | at Hatch | Challenged |
| T01 | Placebo | E18 | 100 ul | In ovo | >75 | 35 | 30 |
| | with MDV | | | vaccine | | | |
| T02 | Placebo | E18 (MDV), | 100 ul | In ovo | >75 | 35 | 30 |
| | with MDV, | D0-56 | vaccine, | vaccine, | | | |
| | Probiotic | (Probiotic) | Probiotic | Water | | | |
| | | | TBD | Treatment | | | |
| T03 | Salmonella | E18 (MDV), | 100 ul | In ovo | >75 | 35 | 30 |
| | with MDV, | D0-56 | vaccine, | vaccine, | | | |
| | Probiotic | (Probiotic) | Probiotic | Water | | | |
| | | | TBD | Treatment | | | |
| T04 | Salmonella | E18 | 100 ul | In ovo | >75 | 35 | 30 |
| | with MDV | | | | | | |

TABLE 3
Sentinel Bird Sampling Schedule

| Group | | |
|---|---|---|
| # | Treatment | Sentinel Birds at D28* |
| T01 | Placebo with MDV | 5 |
| T02 | Placebo with MDV, Probiotic | 5 |
| T03 | Salmonella with MDV, Probiotic | 5 |
| T04 | Salmonella with MDV | 5 |

*Birds will be euthanized, and liver/spleen and cecum tested to confirm negative status for Salmonella prior to challenge (D28).

Throughout this application, regarding age at administration, “E” refers to embryonic (unhatched) age (e.g. E18 refers to embryonic age, day 18), and “D” refers to the age of a chick once hatched, from Day 0 (day of hatching) onward.

### Experimental Subject

The experimental subject is the chicken.

### Allotment/Randomization

Randomization procedure will be conducted by laboratory personnel using the RAND function in Excel and random selection. Eggs will be randomly selected and placed in incubator trays. There will be at least 75 eggs for each treatment group for in ovo vaccination. Treatment details are listed in Table 2. After in ovo vaccination T01 and T02 will be hatched in the same hatcher while T03 and T04 will be hatched in a second hatcher. The hatchers will be in separate rooms.

Tag numbers will be randomized to Groups T01-T04 (35 tag numbers/group) prior to hatch using the RAND function in excel. Healthy chickens will be selected, tagged, and placed in the appropriate isolator based treatment group. A chore order will be assigned to maintain biosecurity. On Day 28, five randomly selected birds from each group will be euthanized and have liver, spleen, and cecum samples collected. All liver/spleen and cecum samples will be tested for Salmonella presence to confirm negative status. See Table 3 for sentinel bird sampling schedule.

### Masking

This will be a partially-blinded study. Any personnel performing laboratory assays will be masked to the allocation of animals to treatments. The treatment administrator will be blinded to treatments. Personnel performing daily husbandry tasks and observations will only be partially blinded due to chore order to maintain adequate biosecurity.

### Schedule of Events

A schedule of events is provided in Table 4.

TABLE 4
Schedule of Events

| Approximate | |
|---|---|
| Study Day | Study Activity |
| E0* | Set eggs |
| E7, E14, E18 | Candling |
| D-3 (E18) | In ovo vaccination |
| | Transfer eggs to hatchers |
| D0 | Begin daily probiotic in water treatment |
| | Hatch, tag, and placement |
| | Individual weights |
| D1-D49 | General Health Observations |
| D28 | Cloacal swabs |
| | Individual weights |
| | Cull and necropsy 20 birds (5 per treatment |
| | group) - liver, spleen, and cecum collected |
| D35 | Challenge |
| | Irradiated litter placed in isolators |
| D56 | Individual weights |
| | Necropsy - liver, spleen, and cecum collected.. |

*Eggs will be set the evening before E0 to allow 18.5 days of incubation

### Investigational Veterinary and Control Products

### IVP

Salmonella High-Dose Vaccine in this Example is an inactivated bacterial vaccine that is made of seven different species/strains of bacteria. These bacterial strains are: Salmonella Enteritidis, Salmonella Typhimurium, Salmonella Agona, Salmonella Kentucky, Pseudomonas Aeuroginosa, Aerobacter Aerogenes, and Escherichia coli. Bacteria may be obtained from Benchmark Biolabs (Lincoln, Nebr., USA) and initially acquired from ATCC (American Type Culture Collection, Manassas, Va., USA). This vaccine is prepared by growing each bacterial strain separately to the desired concentration as in Table 5, followed by separate heat inactivation of each bacterial strain by double autoclaving at 121° C. for 30 min each. Epitope density may be evaluated after heat inactivation. The inactivated bacterial strains will be mixed and transferred to a new container. Both Gentamycin and Nystatin are added accordingly to the neutralized killed vaccine as preservatives. 2× of the final inactivated vaccine will be mixed 1:1 with 2×MDV at the day of inoculation to be used for the corresponding treatment group. Placebo vials that contain DPBS, and Gentamycin and Nystatin at the same concentration as the final vaccine will also be used for the corresponding treatment group.

TABLE 5
Final Concentrations of the High-Dose
Salmonella Vaccine (Salmogenics Vaccine)

| | High dose | % CFU/high dose |
|---|---|---|
| Additive | (CFU/dose) | (vs. total bacteria) |
| S. Kentucky | 3 × 10¹⁰ | 15% |
| S. typhimurium | 4.8 × 10¹⁰ | 24% |
| S. agona | 1.7 × 10¹⁰ | 9% |
| S. enteritidis | 1.8 × 10¹⁰ | 9% |
| E. coli | 1.2 × 10¹² | 6% |
| A. aerogenes | 2.2 × 10¹⁰ | 11% |
| P. aeruginosa | 5.2 × 10¹⁰ | 26% |
| Gentamycin | 30 μg/ml | — |
| Nystatin | 30 μg/ml | — |

### Control Products

In Ovo Placebo Control
PBS (Phosphate-Buffered Saline) with Gentamycin and Nystatin
Labeling/Storage Conditions: 2-8° C.

Marek's Disease Vaccine
Marek's Disease Vaccine (MDV)
Labeling/Storage Conditions: liquid nitrogen
Manufacturer: Merial (Boehringer Ingelheim Animal Health USA, Duluth, Ga.)

Probiotic Treatment
Product Name: Ralco's Strong Animals Flock Fixer™, Hydrate & Restore
Product Total Bacteria Count: Bacillus subtilis, Bifidobacterium longum, Lactobacillus acidophilus (1.007×10⁸ Colony Forming Units (CFU)/g total) Manufacturer: RALCO NUTRITION, INC. (Marshall, Minn., USA)
Preparation in drinking water: 17 g Probiotic Treatment/gallon drinking water

Additional product information: Ralco's Strong Animals Flock Fixer™ Hydrate & Restore includes a minimum of 1.10% salt and a maximum of 1.60% salt, and a minimum of 454 IU/pound Vitamin E. Ingredients: maltodextrin, dextrose, citric acid, potassium chloride, silicon dioxide, hemicellulose extract, sodium chloride, dried Bacillus subtilis fermentation product, organic origanum oil, vitamin E supplement, dried Bifidobacterium longum fermentation product, dried Lactobacillus acidophilus fermentation product, vitamin A supplement, niacin supplement, vitamin D3 supplement, menadione sodium bisulfite complex, calcium pantothenate.

### Challenge Products

Challenge products according to this Example are described in Table 6 below.

TABLE 6
Challenge Products

| | |
|---|---|
| Material Name | Salmonella enterica serovar Heidelberg |
| Material Origin | RTI, LLC (Brookings, SD) |
| Storage Conditions | Frozen ≤−70° C. |
| Identification | Standard Quality Control testing |
| Dosage | Approximately 1 × 10⁵ |
| Route | Oral Gavage |
| Amount Administered | 250 μl |
| Testing/Back Titration | A pre- and post-inoculation titration will |
| | be performed on the challenge material. |

Challenge will be administered via a 250 μl oral gavage to all chickens on D35, as shown in Table 4.

TABLE 7
Animals

| | |
|---|---|
| Animal Ownership | Animals will be owned by RTI, LLC. (Brookings, SD) |
| Species/Breed/Strain | Chicken, White Leghorn |
| Serological/Disease Status | SPF (specific-pathogen-free) |
| Sex | Straight run “as hatched”, males and females not |
| | distinguished |
| Origin | Charles River Laboratories (Norwich, CT) |
| Age or Age Range at Day 0 | Day of hatch |
| (D0) | |
| Identification Method | Neck tags and/or Leg tags |
| Inclusion/Exclusion Criteria | Only healthy chicks will be included in the study and |
| (i.e., to be eligible for | assessed at hatch. Enrolled chicks will be documented on |
| enrollment into the study) | the RTI Hatched Chick Arrival Form (RTI, LLC, Brookings, SD, |
| | USA). |
| Post-inclusion Removal | Chickens will be removed from the study if the Clinical |
| Criteria | Veterinarian or designee deems necessary. For animals |
| | removed or found dead prior to the end of the study, a |
| | general necropsy should be performed and documented |
| | on the appropriate RTI Form. The Study Monitor and |
| | Investigator will be notified. Disposition of the animals will |
| | be recorded on the appropriate RTI Form. |
| Housing | Animals will be housed by treatment group within 4 |
| | isolators. Irradiated litter will be added to the 4 isolators |
| | on the day of challenge. With the exception of stocking |
| | density, which will be reviewed by RTI's Institutional |
| | Animal Care and Use Committee (IACUC) for approval, |
| | animal husbandry and care will be consistent with site |
| | SOPs and the Guide for the Care and Use of Agricultural |
| | Animals in Research and Teaching (Ag Guide), 4^{th} edition, |
| | 2020. Feed, water, and environmental conditions will be |
| | checked at least once daily and documented. |
| Environmental Control | Animal housing temperatures and humidity will be |
| | monitored once daily and documented. |
| Feed and Water | Animals will be fed age-appropriate irradiated feed ad |
| | libitum. Animals will have water ad libitum. |
| Biosecurity | The environment, litter, and feed will be screened for |
| | Salmonella prior to bird exposure. Proper PPE will be used |
| | during this study. |
| Concomitant Therapies | Application of topical treatments may be allowed to |
| | discourage feather pecking or protect minor wounds if the |
| | Investigator or Clinical Veterinarian deems it necessary, |
| | provided it does not interfere with study outcomes. All |
| | treatments will be documented appropriately. |
| Unscheduled Necropsy | Ill animals should be identified to the Veterinarian and, if |
| | necessary in the opinion of the Veterinarian and Principal |
| | Investigator, animals should be humanely euthanized using |
| | an AVMA-approved method. Standard veterinary practices |
| | and disposition will be documented on facility forms. A |
| | general necropsy will be performed within 72 hours of |
| | discovery in an attempt to determine the probable cause. |

TABLE 8
Procedures

| Study Activity | Description |
|---|---|
| General Health | Animals will be observed daily and any abnormal health observations will |
| Observations | be recorded and reported to the Principal Investigator and Clinical |
| | Veterinarian for further evaluation. |
| Litter | Litter will be added to the isolators on day of Challenge. Litter will be |
| Observations | observed at least daily and any abnormal observations will be recorded in |
| | the General Health Observations form. |
| Vaccination | On E18, eggs will be vaccinated in ovo with 100 μl of the appropriate |
| | treatment and documented on an administration record. See Table 2. |
| Probiotic | On D0-D56, chicks in T02 and T03 will receive a probiotic water |
| Treatments | treatment containing a proprietary blend of probiotics and prebiotics. |
| | Details of the final dosage will be described in the final report and |
| | documented on an administration record. |
| Animal Weight | Pen weights will be obtained on D0 prior to placement into isolators. |
| | Individual chicks will be weighed on D28 and D56 prior to necropsy. |
| Cloacal Swabs | On D28, cloacal swabs will be collected from all chicks to test for |
| | presence of Salmonella. |
| Challenge | On day of challenge, birds will be administered 250 μl of challenge |
| | material by oral gavage. |
| Necropsy | Table 3 details sampling schedule for sentinel birds on D28, when birds |
| | will be euthanized and liver, spleen, and cecum will be collected and |
| | tested for Salmonella presence. On day of necropsy (D56), all remaining |
| | birds will be weighed individually and euthanized. Liver, spleen, and |
| | cecum will be collected from each bird and tested for Salmonella |
| | presence. |

### Sample Collection, Processing, and Testing

All samples will be individually identified by label including study number, animal ID, sample type, and date.

TABLE 9
Sample Collection, Processing, and Testing

| | Day of | |
|---|---|---|
| Sample | Collection | Test |
| Cloacal Swabs | Day 28 | Salmonella |
| | | Presence |
| Liver, Spleen, | Days 28 | Salmonella |
| Cecum | and 56 | Presence |

Cloacal Swabs: Swabs from individual animals will be placed into tubes containing 5 mL of tetrathionate brilliant green enrichment broth (TTBG). Tubes will be vortexed and incubated overnight at 42° C. for a total of 48 hours. After 18-24 hours of incubation, samples will be streaked onto Hektoen Enteric (HE) agar and incubated at 37° C. overnight. Following incubation, the HE plates will be examined for the presence of black or black-centered colonies that are typical for Salmonella. Cultures that are negative for colonies typical of Salmonella after being streaked onto HE for the first time will be re-streaked onto HE at approximately 48 hours post-incubation, and the plates evaluated 18-24 hours later.

Tissue Samples: Approximately two grams (g) each of each tissue will be collected and minced/stomached in a 20 ml aliquot of TTBG, which will then be incubated at 37° C. for liver/spleen samples and 42° C. for cecum samples for a total of 48 hours. Liver and spleen samples will be pooled together for analysis.

After 24 hours incubation, an aliquot of each TTBG broth culture will be streaked onto HE agar and incubated at 37° C. for 24 hours. Following 24 hours incubation, the HE plates will be examined for the presence of black or black-centered colonies that are typical for Salmonella. Broth cultures that are negative for colonies typical of Salmonella after being streaked onto HE for the first time will be re-streaked onto HE at 48 hours post-incubation, and the plates will be evaluated 24 hours later.

### Disposition

All animals will be euthanized and disposed of following site-specific disposal procedures and documented. After vaccination, all empty and partial vials of all IVP and CP will be disposed of at RTI according to site procedures. A 1 ml sample may be collected from each IVP and CP and retained. After challenge, a 1 mL sample will be collected and retained. The sample may be returned to the laboratory for confirmation of CFU counts. All remaining challenge material and also sample material will be disposed of following site-specific disposal procedures.

### Effectiveness of Outcome

Referring to Table 2, Salmonella in ovo vaccinated birds (T03, T04) will have less isolation of the challenge strain when compared to unvaccinated controls. Salmonella in ovo vaccinated and probiotic-supplemented birds (T03) will have less isolation of the challenge strains when compared to the unvaccinated controls.

### Data Summary and Analysis

### Statistical Methods

Proportion of tissues (liver and spleen, cecum, cloacal swabs) showing S. Heidelberg recovery (positive vs negative recovery) as a percentage of each treatment group will be determined for comparison.

### Adverse Events

An Adverse Event (AE) is any observation relating to animals that is unfavorable and unintended and occurs after the use of an IVP, whether or not it is considered to be product related. A mild AE, usually transient, requires no specific treatment and generally does not interfere with usual activities. A moderate AE is sufficiently discomforting to interfere with normal activities. Signs may be ameliorated by simple therapeutic measures. A serious AE is any harmful and unintended response resulting in death, life-threatening, persistent, or significant disability/incapacity associated with the use of an IVP, whether or not it is considered to be product related. Discontinuation of the treatment with the IVP is mandatory if the event is life-threatening and evaluations made to suggest a “possible”, “probable”, or “certain” relationship to the IVP. A serious unexpected adverse drug experience is unexpected. It is not reported on the product label and includes any event that may be symptomatically and pathophysiologically related to an event listed on the labeling but differs from the event because of greater severity or specificity.

### Example C

### Probiotic Supplementation on Performance and Campylobacter jejuni Load in Broilers Challenged with C. jejuni

This Example is conducted to determine the effects of B. subtilis and B. licheniformis probiotic supplementation on performance, cecal C. jejuni load, and C. jejuni contamination in meat of broilers challenged with C. jejuni.

A total of 450 one-day-old broiler chicks were randomly distributed to five experimental groups. Each treatment was replicated in 6 pens (n=6) with 15 chicks per pen. The five experimental groups are:

1. Control
2. Control+C. jejuni infection
3. Control+C. jejuni infection+100% B. subtilis-B. licheniformis*
4. Control+C. jejuni infection+50% B. subtilis-B. licheniformis*
5. Control+C. jejuni infection+10% B. subtilis-B. licheniformis*
   *100% B. subtilis-B. licheniformis: 10 mg/Kg feed of Probiotic Treatment A+100 mg/kg feed of Probiotic Treatment B
   *50% B. subtilis-B. licheniformis: 5 mg/Kg feed of Probiotic Treatment A+50 mg/kg feed of Probiotic Treatment B
   *10% B. subtilis-B. licheniformis: 1 mg/Kg feed of Probiotic Treatment A+10 mg/kg feed of

### Probiotic Treatment B

Probiotic Treatment A: Bacillus subtilis (1×10¹¹ CFU/g) Powder, 100B, stored <30° C. (Synergia Life Sciences PVT. LTD., Mumbai, India)
Probiotic Treatment B: Powdered Blend of B. licheniformis & B. subtilis natto VBN 101 (1×10⁸ CFU/g), 100M, stored <30° C. (Synergia Life Sciences PVT. LTD., Mumbai, India)

At 14 days of age, birds were individually challenged with 1 ml of 1×10⁸ CFU field strain of C. jejuni via oral gavage.

At 42 days of age, all birds were euthanized.

### Sample Collection:

Samples were collected from one bird per replication (6 birds per treatment) at days 14, 21, 28, 35, and 42 of age. Jejunal section were collected in formalin to evaluate the villi height and crypt depth parameters. Cecal content were frozen for analyzing the total C. jejuni loads by real time PCR at all time points of sample collection as described in attached article. Cecal content were grown in Campy Cefex per standard procedures. Bile and serum were analyzed for anti-Campylobacter IgA.

At 42 days of age, three birds per pen were analyzed for C. jejuni carcass load. Birds and C. jejuni infection

A total of 450 one-day old Cobb-500 broiler chicks were randomly distributed to five experimental groups; Control, C. jejuni infection, C. jejuni infection+Probiotic Treatment B 10 mg/Kg feed, C. jejuni infection+Probiotic Treatment B 50 mg/Kg feed, C. jejuni infection+Probiotic Treatment B 100 mg/Kg feed. Each treatment was replicated in 6 pens (n=6) with 15 chicks per pen. The Control group received a basal diet (Table 1) based on corn and soybean diet that met the minimal NRC requirement. At 21 days of age, all birds in C. jejuni groups were gavaged orally with 1 ml of 1×10⁸ CFU C. jejuni as described previously. Body weight and feed consumption was measured at weekly intervals and body weight gain and feed efficiency was calculated.

Effect of Probiotic Supplementation on Caecal C. jejuni Load Post-C. jejuni Infection in Broiler Birds

At 0, 7, 21, and 28 days post-infection, caecal contents were collected and analyzed for C. jejuni load by plating on the campy-CEFEX agar plates. The data was expressed as log numbers

Effect of Probiotic Supplementation on Bile and Serum Anti-C. jejuni IgA Content Post-Salmonella Infection

Bile and serum samples were collected from one bird per pen at D 21, 28, 35, and 42 days of age and analyzed for anti-C. jejuni IgA content using an enzyme-linked immunosorbent assay (ELISA). The primary and secondary antibody concentrations were established using checkerboard titrations with dilutions of bile, serum and antigens. C. jejuni antigen for coating was made by 3 consecutive freeze thaw cycles of pure culture of C. jejuni followed by mechanical lysing. The pure culture was lysed two times by glass beads size 425-600 μm (Sigma, St. Louis, Mo.) in a TissueLyser LT (Qiagen, Germany) for 5 minutes at 50 Hz. The lysed cells were centrifuged at 10,000×g for 10 min and the resultant supernatant was collected and stored at −70° C. until use. Flat-bottomed 96-well microtitration plates (Microlon 600° High Binding, Greiner, N.C., USA) were coated with 100 μl of 10 μg/ml of the antigen diluted in 0.1M carbonate buffer and incubated overnight at 4° C. The plates were washed three times with PBS-Tween 20. To prevent non-specific binding, wells were blocked with 100 μl of 8% nonfat dry milk-PBS-Tween 20 and incubated for 1.5 h at 37° C. For IgA analysis, 100 μl of 1:200 dilution of the bile in 8% nonfat dry milk-PBS-Tween 20 was added to the plates in duplicates and incubated for 1.5 h at room temperature. For IgG analysis, 100 μl of 1:200 dilution of the serum in 8% nonfat dry milk-PBS-Tween 20 was added to the plates in duplicates and incubated for 1.5 h at room temperature. After washing, 100 μl of 1:100,000 dilutions of HRP-labeled anti-chicken IgA (Novus Biologicals, CO, USA) or HRP-labeled anti-chicken IgG (Novus Biologicals, CO, USA) in PBS-Tween 20-5% nonfat dry milk was added to each well and incubated for 1 h at room temperature. The plates were washed with PBS-Tween 20, and the substrate 3,3,5,5-tetramethylbenzidine (TMB) solution (eBioscience, San Diego, Calif., USA) was added to the wells (100 μl/well). The reaction was stopped after 10 min using 1N HCl (100 μl/well), and the optical density was read at 450 nm using a microplate ELISA reader. IgA values were reported as the mean optical density.

### Effect of Probiotic Supplementation on Jejunum Villus Height, Crypt Depth, and Villus Height to Crypt Depth Ratio

Jejunum samples were collected from one bird per pen at D14, 21. 28, 35, and 42 days of age. A 2 cm section of the jejunum, distal to Meckel's Diverticulum, was removed and placed in 10% formalin solution. The tissues were then dehydrated in a series of graded alcohol and then infiltrated with paraffin overnight at 60° C. Using a microtome, the paraffin blocks were then cross-sectioned into 5 μm sections and mounted on to frosted microscope slides. The slides were warmed to 37° C. and stained with hematoxylin and eosin. Each cross-section was viewed at 100× magnification and photographed using a microscope coupled with a camera. Using ImageJ software, three villi per cross section were measured to determine villi height, crypt depth, and the ratio of villi height to crypt depth.

### Statistical Analysis

A one-way ANOVA was used to examine the effects of probiotic supplementation and C. jejuni challenge on dependent variables post-c. jejuni infection. When the interaction effects were significant (P<0.05), differences between means were analyzed by Tukey's least square means comparison.

### Results

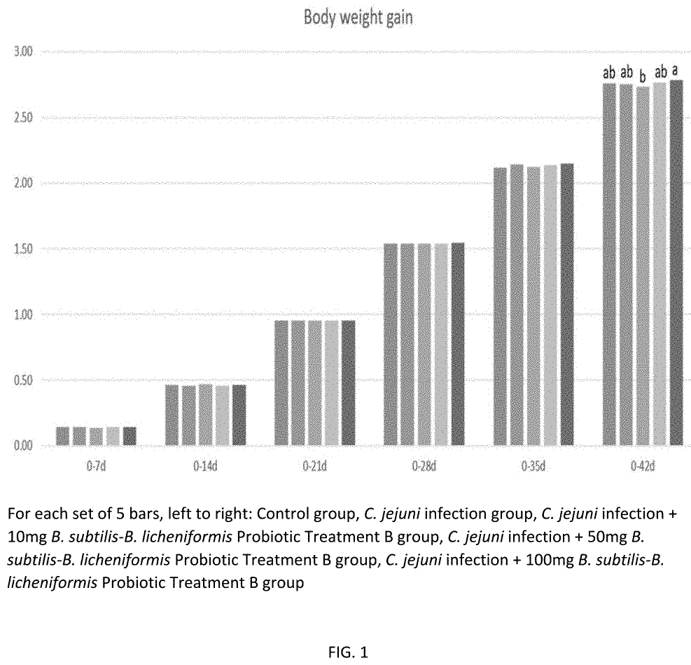

FIG. 1 shows body weight gain (BWG) in chickens at 7 days, 14 days, 21 days, 28 days, 35 days, and 42 days of age (for each set of 5 bars, left to right: experimental group 1. (Control), 2. (Control+C. jejuni infection), 3. (Control+C. jejuni infection+100% B. subtilis-B. licheniformis), 4. (Control+C. jejuni infection+50% B. subtilis-B. licheniformis), 5. (Control+C. jejuni infection+10% B. subtilis-B. licheniformis). There were no significant effects (P>0.05) on BWG between the birds in the treatment groups between 0-35 days of age.

There were significant effects (P<0.05) on day O-42 BWG between the birds in the treatment groups. Birds fed diets supplemented with 100 mg/Kg Probiotic Treatment B had comparable body weight to the control group with no C. jejuni infection and had the highest BWG at day 42.

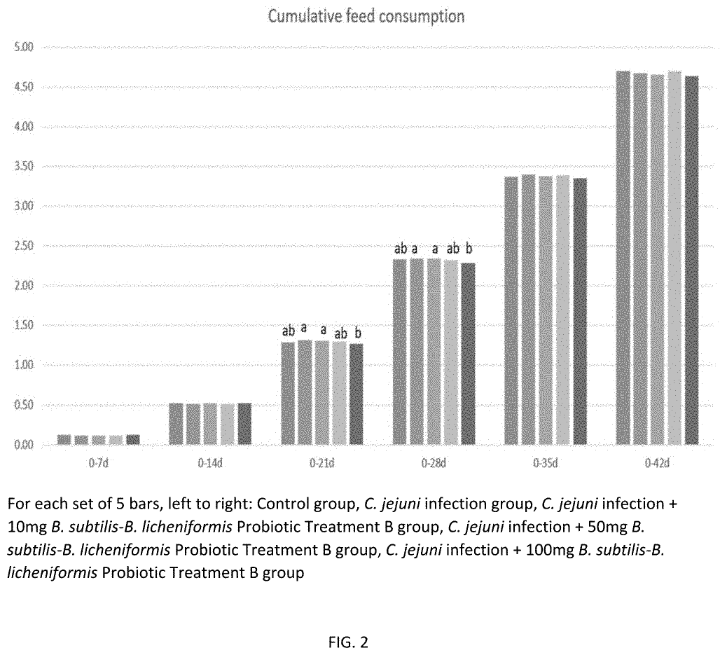

FIG. 2 shows cumulative feed consumption (FC) in chickens at 7 days, 14 days, 21 days, 28 days, 35 days, and 42 days of age (for each set of 5 bars, left to right: experimental group 1. (Control), 2. (Control+C. jejuni infection), 3. (Control+C. jejuni infection+100% B. subtilis-B. licheniformis), 4. (Control+C. jejuni infection+50% B. subtilis-B. licheniformis), 5. (Control+C. jejuni infection+10% B. subtilis-B. licheniformis). There were no significant effects (P>0.05) on FC between the birds in the treatment groups between O-7, 0-14, 0-35, and O-42 days of age.

There were significant effects (P<0.05) on D O-21 and D O-28 FC between the birds in the treatment groups. Birds fed diets supplemented with 100 mg/Kg Probiotic Treatment B and challenged with the C. jejuni had lower O-21 days and O-28 days feed consumption than that in the challenge control group.

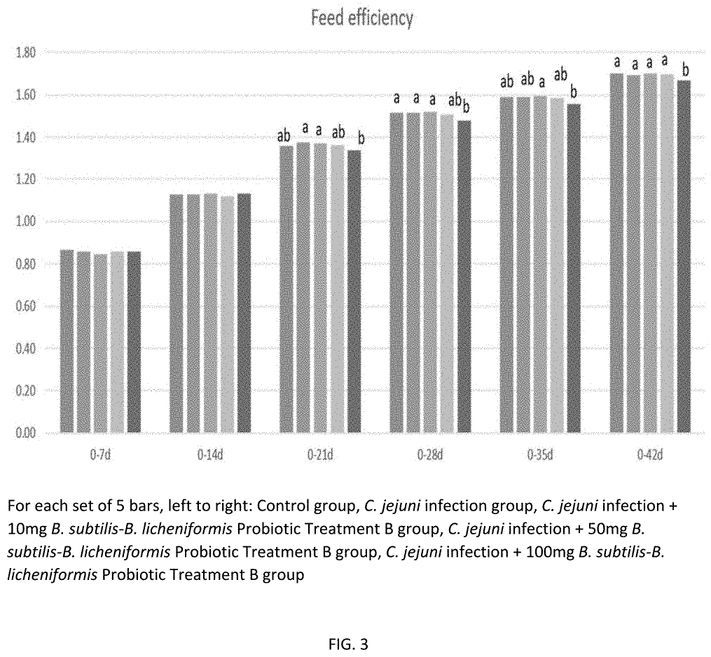

FIG. 3 shows feed efficiency (FEC) in chickens at 7 days, 14 days, 21 days, 28 days, 35 days, and 42 days of age (for each set of 5 bars, left to right: experimental group 1. (Control), 2. (Control+C. jejuni infection), 3. (Control+C. jejuni infection+100% B. subtilis-B. licheniformis), 4. (Control+C. jejuni infection+50% B. subtilis-B. licheniformis), 5. (Control+C. jejuni infection+10% B. subtilis-B. licheniformis). There were no significant effects (P>0.05) on FC between the birds in the treatment groups between O-7, O-14, O-35, and O-42 days of age.

There were no significant effects (P>0.05) on FEC between the birds in the treatment groups between O-7 and O-14 days of age.

There were significant effects (P<0.05) on D O-21, 0-28, 0-35, and O-42 day FEC between the birds in the treatment groups. Birds fed diets supplemented with 100 mg/Kg Probiotic Treatment B and challenged with the C. jejuni had better D O-21, 0-28, 0-35, and O-42 day feed efficiency than that in the challenge control group.

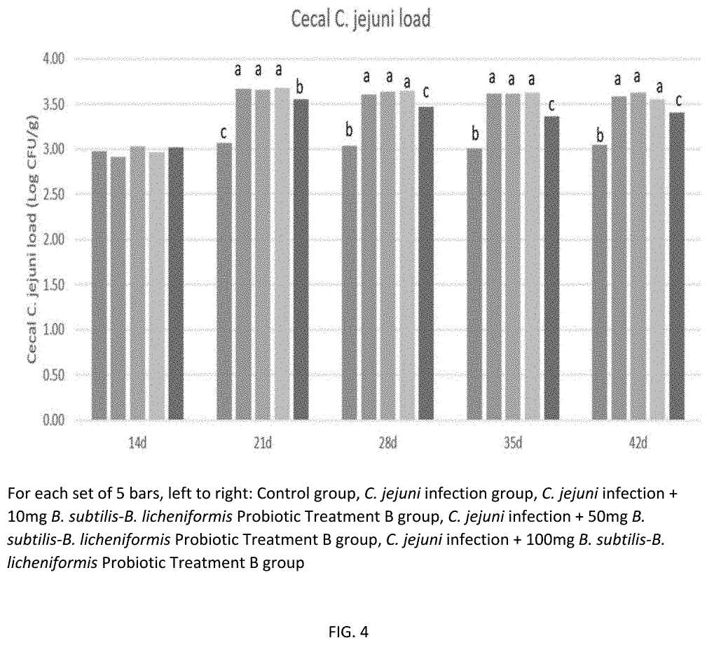

FIG. 4 shows cecal C. jejuni load in chickens at 14 days, 21 days, 28 days, 35 days, and 42 days of age (for each set of 5 bars, left to right: Control group, C. jejuni infection group, C. jejuni infection+10 mg B. subtilis-B. licheniformis group, C. jejuni infection+50 mg B. subtilis-B. licheniformis group, C. jejuni infection+100 mg B. subtilis-B. licheniformis group). There were no significant effects (P>0.05) on cecal C. jejuni loads between the birds in the treatment groups before C. jejuni challenge at 14 d of age.

There were significant effects (P<0.05) on cecal C. jejuni loads at D 21, 28, 35, and 42 days of age between the birds in the treatment groups. Birds challenged with C. jejuni had higher levels of cecal C. jejuni loads compared to the birds in the control group. Birds fed diets supplemented with 100 mg/Kg Probiotic Treatment B and challenged with the C. jejuni had significantly lower cecal C. jejuni loads than that in the challenge control group.

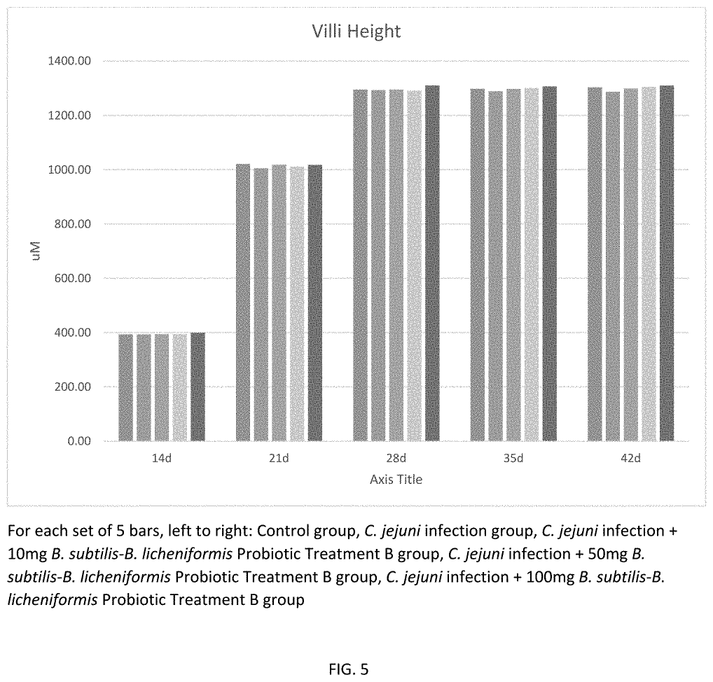

FIG. 5 shows villi height in chickens at 14 days, 21 days, 28 days, 35 days, and 42 days of age (for each set of 5 bars, left to right: Control group, C. jejuni infection group, C. jejuni infection+10 mg B. subtilis-B. licheniformis group, C. jejuni infection+50 mg B. subtilis-B. licheniformis group, C. jejuni infection+100 mg B. subtilis-B. licheniformis group). There were no significant effects (P>0.05) on villi height between the birds in the treatment groups at any of the time points studied.

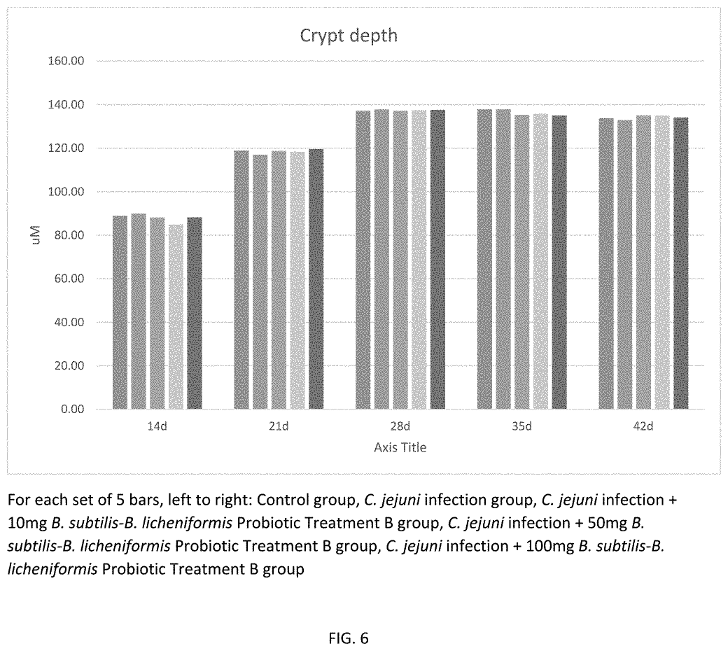

FIG. 6 shows crypt depth in chickens at 14 days, 21 days, 28 days, 35 days, and 42 days of age (for each set of 5 bars, left to right: Control group, C. jejuni infection group, C. jejuni infection+10 mg B. subtilis-B. licheniformis group, C. jejuni infection+50 mg B. subtilis-B. licheniformis group, C. jejuni infection+100 mg B. subtilis-B. licheniformis group). There were no significant effects (P>0.05) on crypt depth between the birds in the treatment groups at any of the time points studied.

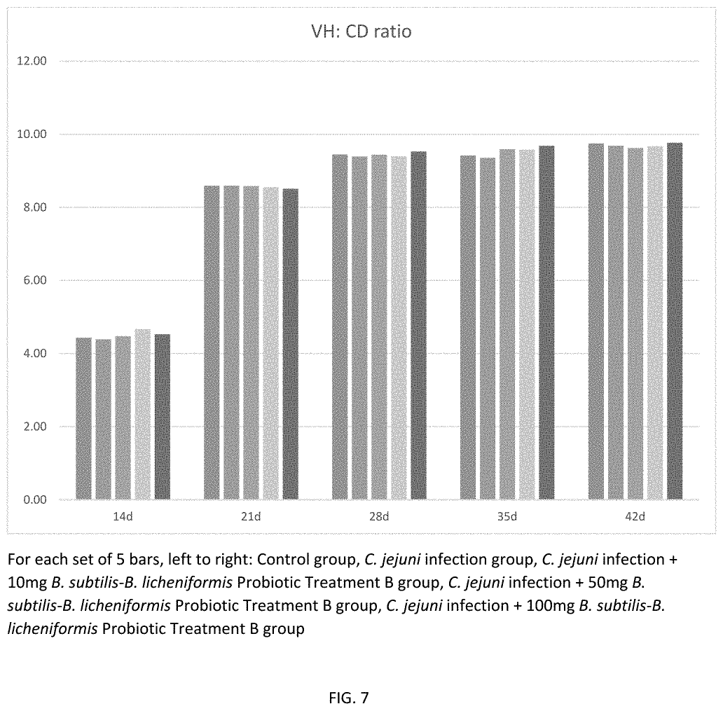

FIG. 7 shows villi height to crypt depth ratio in chickens at 14 days, 21 days, 28 days, 35 days, and 42 days of age (for each set of 5 bars, left to right: Control group, C. jejuni infection group, C. jejuni infection+10 mg B. subtilis-B. licheniformis group, C. jejuni infection+50 mg B. subtilis-B. licheniformis group, C. jejuni infection+100 mg B. subtilis-B. licheniformis group). There were no significant effects (P>0.05) on Villi height: crypt depth ratio between the birds in the treatment groups at any of the time points studied.

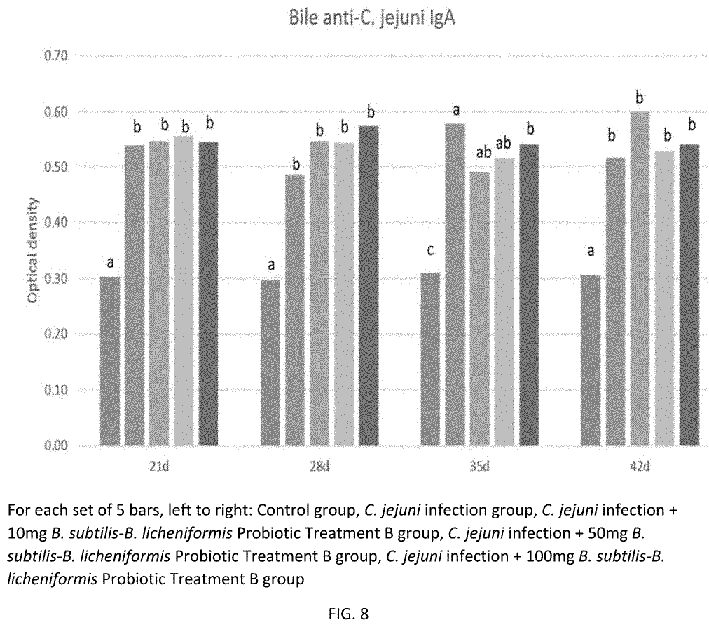

FIG. 8 shows bile anti-C. jejuni IgA in chickens at 21 days, 28 days, 35 days, and 42 days of age (for each set of 5 bars, left to right: Control group, C. jejuni infection group, C. jejuni infection+10 mg B. subtilis-B. licheniformis group, C. jejuni infection+50 mg B. subtilis-B. licheniformis group, C. jejuni infection+100 mg B. subtilis-B. licheniformis group). There were significant effects (P<0.05) on bile anti-C. jejuni specific IgA amounts at D 21, 28, 35, and 42 days of age between the birds in the treatment groups. Birds challenged with C. jejuni had higher levels of anti-C. jejuni specific IgA at all time points studied.

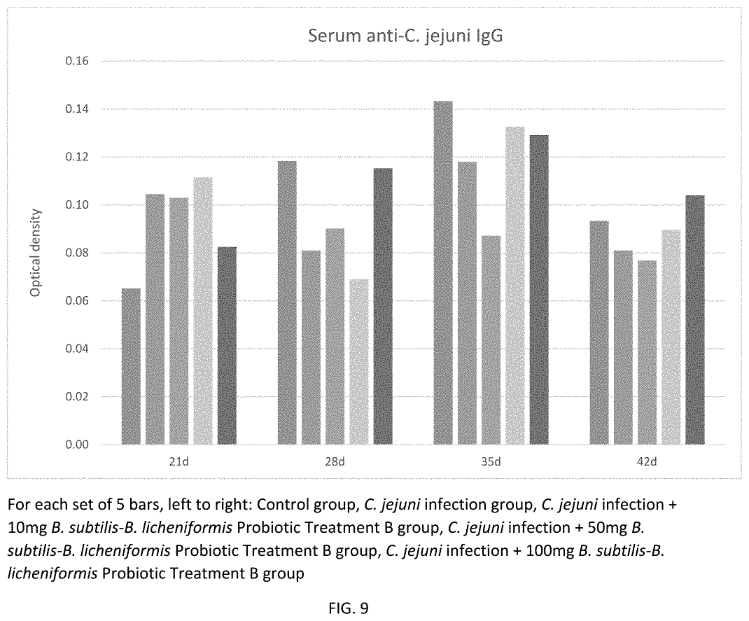

FIG. 9 shows serum anti-C. jejuni IgG in chickens at 21 days, 28 days, 35 days, and 42 days of age (for each set of 5 bars, left to right: Control group, C. jejuni infection group, C. jejuni infection+10 mg B. subtilis-B. licheniformis group, C. jejuni infection+50 mg B. subtilis-B. licheniformis group, C. jejuni infection+100 mg B. subtilis-B. licheniformis group). There were no significant effects (P>0.05) on serum anti-C. jejuni IgG amounts between the birds in the treatment groups at any of the time points studied.

### Example D

### Inactivation and Preparation of a Salmogenics Vaccine and Efficacy and Protection Against Salmonella Challenge with Vaccine and Probiotic Treatment

This Example will show the efficacy of an inactivated Salmonella vaccine against a Salmonella challenge when administered in combination with a probiotic in-water treatment.

TABLE 10
Study Design (Salmonella Challenge at D35, Necropsy at D28 and D56)

| | | | | | | # Chicks | # Chicks |
|---|---|---|---|---|---|---|---|
| Group | | Age at In ovo | Dose | | # eggs | Enrolled | Challenged |
| # | Treatment | Administration | (ul/egg) | Route | Vaccinated | at Hatch | at D35 |
| T01 | Salmogenics | E18 | 100 μl | In ovo | >65 | 35 | 30 (ST) |
| | (ST) | | | | | | |
| T02 | Salmogenics | E18 | 100 μl | In ovo | >65 | 35 | 30 (SE) |
| | (SE) | | | | | | |
| T03 | Salmogenics | E18 | 100 μl | In ovo | >65 | 35 | 30 (ST) |
| | (SE-ST-SK) | | | | | | |
| T04 | Salmogenics | E18 | 100 μl | In ovo | >65 | 35 | 30 (SE) |
| | (SE-ST-SK) | | | | | | |
| T05 | Placebo (ST | E18 | 100 μl | In ovo | >65 | 35 | 30 (ST) |
| | Challenge | | | | | | |
| | Control) | | | | | | |
| T06 | Placebo (SE | E18 | 100 μl | In ovo | >65 | 35 | 30 (SE) |
| | Challenge | | | | | | |
| | Control) | | | | | | |

TABLE 11
Sentinel Bird Sampling Schedule

| Group | | |
|---|---|---|
| # | Treatment | Sentinel Birds at D28* |
| T01 | Salmogenics (ST) | 5 |
| T02 | Salmogenics (SE) | 5 |
| T03 | Salmogenics (SE-ST-SK) | 5 |
| T04 | Salmogenics (SE-ST-SK) | 5 |
| T05 | Placebo (ST Challenge | 5 |
| | Control) | |
| T06 | Placebo (SE Challenge | 5 |
| | Control) | |

*Birds will be euthanized, and liver/spleen and cecum tested to confirm negative status for Salmonella prior to challenge (D28).

Throughout this application, regarding age at administration, “E” refers to embryonic (unhatched) age (e.g. E18 refers to embryonic age, day 18), and “D” refers to the age of a chick once hatched, from Day 0 (day of hatching) onward.

### Experimental Subject

The experimental subject is the chicken.

### Allotment/Randomization

Randomization procedure will be conducted by laboratory personnel using the RAND function in Excel and random selection. Eggs will be randomly selected and placed in incubator trays. There will be at least 75 eggs for each treatment group for in ovo vaccination. Treatment details are listed in Table 2. After in ovo vaccination T05 and T06 will be hatched in a second hatcher. The hatchers will be in separate rooms.

Tag numbers will be randomized to Groups T01-T04 (35 tag numbers/group) prior to hatch using the RAND function in excel. Healthy chickens will be selected, tagged, and placed in the appropriate isolator based treatment group. A chore order will be assigned to maintain biosecurity. On Day 28, five randomly selected birds from each group will be euthanized and have liver, spleen, and cecum samples collected. All liver/spleen and cecum samples will be tested for Salmonella presence to confirm negative status. See Table 11 for sentinel bird sampling schedule.

### Masking

This will be a partially-blinded study. Any personnel performing laboratory assays will be masked to the allocation of animals to treatments. The treatment administrator will be blinded to treatments. Personnel performing daily husbandry tasks and observations will only be partially blinded due to chore order to maintain adequate biosecurity.

### Schedule of Events

A schedule of events is provided in Table 12.

TABLE 12
Study Events

| Study Activity | Description |
|---|---|
| In ovo Vaccination | On E18, eggs will be vaccinated in ovo with |
| | 100 ul of the appropriate treatment and |
| | documented on an administration record. |
| Body Weight | Pen weights will be obtained on D0 prior to |
| | placement into isolators. Individual chicks |
| | will be weighed on D28 and D56 prior to |
| | necropsy. |
| Cloacal Swabs | On D28, cloacal swabs will be collected from |
| | all chicks to test for presence of Salmonella. |
| Salmonella Challenge | On day of challenge (D35), birds will be |
| | administered 250 ul of challenge material (SE |
| | or ST) by oral gavage. |
| Necropsy | On D28, 5 birds from each treatment group |
| | will be necropsied. Liver, spleen, and cecum |
| | will be collected from each bird and tested |
| | for presence of Salmonella. |
| | On D56, all remaining birds from each |
| | treatment group will be necropsied. Liver, |
| | spleen, and cecum will be collected from |
| | each bird and tested for presence of SE or ST. |

### Investigational Veterinary and Control Products

### IVP

A Salmogenics vaccine in this Example is an inactivated bacterial vaccine that is made of seven different species/strains of bacteria. The vaccine comprises: Salmonella enterica subspecies (subsp.) enterica serovar Enteritidis (Salmonella Enteritidis; SE), Salmonella enterica subsp. enterica serovar Typhimurium (Salmonella Typhimurium; ST), Salmonella enterica subsp. enterica serovar Agona (Salmonella Agona), Salmonella enterica subsp. enterica serovar Kentucky (Salmonella Kentucky; SK), Pseudomonas Aeuroginosa, Aerobacter Aerogenes AHP462/PTA-11661, and Escherichia coli isolate #7. Bacteria are obtained from Benchmark Biolabs (Lincoln, Nebr., USA) and initially acquired from ATCC (American Type Culture Collection, Manassas, Va., USA). This vaccine is prepared by growing each bacterial strain separately to the desired concentration as in Table 13, followed by separate heat inactivation of each bacterial strain by double autoclaving at 121° C. for 30 min each. Epitope density may be evaluated after heat inactivation. Generally speaking, denatured proteins have lower epitope density and are less immunogenic. Each bacterial protein may be extracted after heat-inactivation and tested by SDS-PAGE in comparison with a protein from the control bacterial strain. The results may be used to provide quality control for in ovo vaccines of this invention. The inactivated bacterial strains will be mixed and transferred to a new container. Both Gentamycin and Nystatin are added accordingly to the neutralized killed vaccine as preservatives. 2× of the final inactivated vaccine will be mixed 1:1 with 2×MDV at the day of inoculation to be used for the corresponding treatment group. Placebo vials that contain DPBS, and Gentamycin and Nystatin at the same concentration as the final vaccine are also prepared to be used for the corresponding treatment group.

TABLE 13
Final Concentrations of the Salmogenics Vaccine

| | High dose | % CFU/high dose |
|---|---|---|
| Additive | (CFU/dose) | (vs. total bacteria) |
| S. Kentucky | 3 × 10¹⁰ | 15% |
| S. typhimurium | 4.8 × 10¹⁰ | 24% |
| S. agona | 1.7 × 10¹⁰ | 9% |
| S. enteritidis | 1.8 × 10¹⁰ | 9% |
| E. coli | 1.2 × 10¹² | 6% |
| A. aerogenes | 2.2 × 10¹⁰ | 11% |
| P. aeruginosa | 5.2 × 10¹⁰ | 26% |
| Gentamycin | 30 μg/ml | — |
| Nystatin | 30 μg/ml | — |

### Control Products

In Ovo Placebo Control

PBS (Phosphate-Buffered Saline) with Gentamycin and Nystatin

### Labeling/Storage Conditions: 2-8° C.

Sample Collection, Processing, and Testing

Cloacal Swabs: Swabs from individual birds will be placed into tubes containing 5 mL of tetrathionate brilliant green (TTBG) enrichment broth. Tubes will be vortexed and incubated overnight at 42° C. for a total of 48 hours. After 18-24 hours of incubation, samples will be streaked onto Hektoen Enteric (HE) agar and incubated at 37° C. overnight. Following incubation, the HE plates will be examined for the presence of black or black-centered colonies that are typical for Salmonella. Cultures that are negative for colonies typical of Salmonella after being streaked onto HE for the first time will be re-streaked onto HE at approximately 48 hours post-incubation, and the plates evaluated 18-24 hours later.

Tissue Samples: Approximately two grams (g) of each tissue will be collected and minced/stomached in a 20 ml aliquot of TTBG, which will then be incubated at 37° C. for liver/spleen samples and 42° C. for cecum samples for a total of 48 hours. Liver and spleen samples will be pooled together for analysis. After 24 hours incubation, an aliquot of each TTBG broth culture will be streaked onto HE agar and incubated at 37° C. for 24 hours. Following 24 hours incubation, the HE plates will be examined for the presence of black or black-centered colonies that are typical for Salmonella. Broth cultures that are negative for colonies typical of Salmonella after being streaked onto HE for the first time will be re-streaked onto HE at 48 hours post-incubation, and the plates will be evaluated 24 hours later.

The use of the terms “a,” “an,” “the,” and similar referents in the context of describing the present invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Use of the terms “about” and “substantially” is intended in an embodiment to describe values either above or below the stated value in a range of approximately ±20%; in other embodiments, the values may range in value above or below the stated value in a range of approximately ±5%; in other embodiments, the values may range in value above or below the stated value in a range of approximately ±2%; in other embodiments, the values may range in value above or below the stated value in a range of approximately ±1%. The preceding ranges are intended to be made clear by context, and no further limitation is implied. All method steps described herein may be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise stated. No language in the specification should be construed as indicating any non-claimed element as essential to the practice of the invention.

While in the foregoing specification the present invention has been described in relation to certain embodiments thereof, and many details have been put forth for the purposes of illustration, it will be apparent to those skilled in the art that the invention is susceptible to additional embodiments and that certain of the details described herein may be varied considerably without departing from the basic principles of the invention.

The present invention may be embodied in other specific forms without departing from the spirit or essential attributes thereof, and, accordingly, reference should be made to the appended claims, rather than to the foregoing specification, as indicating the scope of the invention.

1. A product comprising a combination of an in ovo vaccine and a probiotic composition.

2. The product of claim 1, wherein said in ovo vaccine comprises Salmonella bacteria, antigens, and/or epitopes.

3. The product of claim 2, wherein said Salmonella bacteria comprises Salmonella enterica.

4. The product of claim 3, wherein said Salmonella enterica is at least one of subspecies enteritidis, typhimurium, agona, and Kentucky.

5. The product of claim 1, wherein said in ovo vaccine comprises Salmonella Kentucky, Salmonella typhimurium, Salmonella agona, Salmonella enteritidis, Escherichia coli, Aerobacter aerogenes, and Pseudomonas aeruginosa.

6. The product of claim 1, wherein said in ovo vaccine is a discrete dosage unit comprising about 100 million (10⁸) to about 10 trillion (10¹³) CFU.

7. The product of claim 6, wherein said in ovo vaccine is a bacterin vaccine.

8. The product of claim 7, wherein said in ovo vaccine includes about 10¹⁰ to about 10¹² CFU in total.

9. The product of claim 1, wherein said probiotic comprises at least one of Bacillus amyloliquefaciens, B. licheniformis, B. subtilis, B. pumulis, Lactobacillus reuteri, L. acidophilus, L. rhamnosus, and Saccharomyces cerevisiae.

10. The product of claim 9, wherein said probiotic comprises 25 to 30 billion CFU/ml of said bacteria and/or yeast.

11. The product of claim 9, wherein said probiotic comprises a dosage amount of about 7-13 million CFU.

12. The product of claim 11, wherein said dosage amount is about 9-11 million CFU.

13. The product of claim 11, wherein said dosage amount is about 10 million CFU.

14. The product of claim 9, wherein said probiotic is in the form of a discrete dosage unit.

15. The product of claim 9, wherein said probiotic is formulated for oral administration.

16. The product of claim 9, wherein said probiotic is formulated in a solid dosage form.

17. The product of claim 9, wherein said product is water-soluble.

18. The product of claim 1, wherein said combination is for reducing or preventing infection in an avian and/or for reducing or preventing transmission of infection to or from an avian.

19. The product of claim 1, wherein said combination comprising an in ovo vaccine and a probiotic composition is a kit.

20. The product of claim 16, wherein said kit further comprises instructions for administering the combination.

21. A method of treating or preventing infection in an avian subject comprising the steps of
(1) administering an in ovo vaccine to the subject, and
(2) after hatching, administering a probiotic composition to the subject.

22. The method of claim 21, wherein said in ovo vaccine is a bacterin vaccine comprising Salmonella Kentucky, Salmonella typhimurium, Salmonella agona, Salmonella enteritidis, Escherichia coli, Aerobacter aerogenes, and Pseudomonas aeruginosa.

23. The method of claim 22, wherein said vaccine is a high dose Salmonella vaccine.

24. The method of claim 21, wherein said probiotic comprises at least one of Bacillus amyloliquefaciens, B. licheniformis, B. subtilis, B. pumulis, Lactobacillus reuteri, L. acidophilus, L. rhamnosus, and Saccharomyces cerevisiae in a total amount of about 9 to 11 million CFU per dose.

25. The method of claim 21, wherein said probiotic is administered on the day the avian hatches or within 1, 2, 3, or 4 days of hatching.

26. The method of claim 25, wherein the probiotic is administered for at least 3 days.

27. The method of claim 21, wherein the probiotic is administered orally.

28. The method of claim 21, wherein the probiotic is water-soluble.

29. The method of claim 28, wherein the probiotic is administered in drinking water.

30. The method of claim 21, wherein said method reduces or prevents infection in an avian.

31. The method of claim 21, wherein said method reduces or prevents transmission of infection to or from an avian.
